(19) United States
(12) Patent Application Publication
(10) Pub. No.: US 2005/0074829 A1
(43) Pub. Date: Apr. 7, 2005

(54) Methods for identifying inhibitors of sterol 14-alpha-demethylase

(76) Inventors: Abubakr Aslamkhan, Durham, NC (US); Lining Guo, Chapel Hill, NC (US); John W. Rice, Pittsboro, NC (US)

Correspondence Address: Icoria, Inc., 108 T.W. ALEXANDER DRIVE, P O BOX 14528, RTP, NC, 27709-4528, US

(21) Appl. No.: 10/678,571

(22) Filed: Oct. 3, 2003

(51) Int. Cl. C12Q 1/48

(52) U.S. Cl. 435/15

(57) ABSTRACT

In the methods of the present invention, the change in absorbance of an obtusifoliol 14α-demethylase (OBT-DM) enzyme upon binding of an inhibitor is used to advantage for the identification of inhibitors of OBT-DM activity. Rather than measuring the absorbance of an OBT-DM/inhibitor complex over a spectrum of wavelengths as described previously, the present invention discloses methods for identifying type II inhibitors of OBT-DM by monitoring the absorbance only at 413 nm and 432 nm. The methods of the invention enable the concurrent testing of multiple compounds using a high throughput format such as with 96- or 384-well plates. The OBT-DM polypeptides of the invention include plant, fungal and human OBT-DM polypeptides, and in particular, Arabidopsis thaliana OBT-DM polypeptide

### FIELD OF THE INVENTION

The invention relates to methods for the identification of inhibitors of sterol 14-alpha-demethylase. The methods of the present invention are amenable for use in high throughput formats.

### BACKGROUND OF THE INVENTION

Obtusifoliol 14α-demethylase (OBT-DM), also known as CYP51, catalyzes the oxidative cleavage of the methyl group at the 14^{th} position of the steroid ring (Kushiro et al., 285 Biochem. Biophys. Res. Commum. 98-104 (2001)) (see FIG. 1). The product of this reaction is essential for the biosynthesis of steroids in plants, animals, and fungi (Kushiro et al., supra). OBT-DM belongs to the cytochrome P450 family of proteins (Bak et al., 11 Plant J, 191-201 (1997)). Cytochrome P450 proteins contain a heme moiety at the catalytic site with its central iron atom coordinated to the thiol group (FIG. 2) of the side chain cysteine residue that is conserved in all members of the P450 family (Podust et al., 98 Proc. Natl. Acad. Sci. 3068-73 (2001)).

Due to its pivotal role in steroid metabolism, CYP51 has attracted a great deal of attention towards the development of compounds that act as inhibitors of its activity. This is particularly true for the fungal enzyme (Ji et al., 43 J. Med. Chem. 2493-505 (2000); Hitchcock, 19 Biochem. Soc. Trans. 782-7 (1991); Venkateswarlu et al., 40 Antimicrob. Agents Chemother. 1382-6 (1996); Ji et al., 46 J. Med. Chem. 474-85 (2003)). However, recent attempts have also been made to find inhibitors for plant CYP51 enzyme based on knowledge gained from its fungal orthologue (Sekimata et al., 50 J. Ag. Food Chem. 3486-90 (2002); Lamb et al., 284 Biochem. Biophys. Res. Commun. 845-9 (2001)).

Commercial CYP51 inhibitors are based on a triazole or imidazole moiety (FIG. 3A-J). The nitrogen of these five-member heterocyclics coordinate with heme iron as a sixth ligand causing the latter to shift from high to low spin state, which is reflected by an accompanying red shift in the Soret band (FIG. 2A-C). The degree of the shift is a measure of the strength of interaction between the inhibitor compound and the OBT-DM protein. The difference spectrum (spectrum before the addition of the inhibitor subtracted from the one taken after the addition) shows a trough at 413 nm and a peak at 432 nm and is referred to as a “type II spectrum” (Lamb et al., supra) to distinguish it from the spectrum obtained as a result of binding of the substrate to the enzyme or “type I spectrum” (Bak et al., supra).

The present invention discloses methods for identifying compounds that are inhibitors of OBT-DM, in particular, compounds that function as herbicides.

### SUMMARY OF THE INVENTION

The present invention is directed to methods for identifying inhibitors of an OBT-DM enzyme by incubating an OBT-DM polypeptide in the presence and absence of a test compound under conditions suitable for OBT-DM activity, and measuring the amplitude of the difference between the absorbance at 432 nm and the absorbance at 413 nm in the presence and the absence of the test compound, wherein an increase in the amplitude in the presence of the test compound indicates that the compound is an OBT-DM inhibitor. In the methods of the present invention, the change in absorbance of an OBT-DM protein upon binding of a type II inhibitor is used to advantage to identify inhibitory compounds. Rather than measuring the absorbance of an OBT-DM/inhibitor complex over a spectrum of wavelengths as described previously, the present invention discloses methods for identifying type II inhibitors of OBT-DM by monitoring the absorbance only at 413 nm and 432 nm. Thus, the methods of the present invention enable the concurrent testing of multiple compounds using a high throughput format such as with 96- or 384-well plates. The OBT-DM polypeptides of the invention include plant, fungal and human OBT-DM polypeptides, and in particular, Arabidopsis thaliana OBT-DM polypeptide.

### BRIEF DESCRIPTION OF THE FIGURES

FIG. 1. Diagram of the reaction catalyzed by obtusifoliol 14α-demethylase (OBT-DM). The enzyme catalyzes the 14α demethylation of sterols to produce the corresponding 8,14-dienes. Demethylation of obtusifoliol by OBT-DM requires the participation of cytochrome P450 reductase, which reduces the heme iron from ferric (resting) to ferrous state, thereby allowing the reduction of the bound oxygen molecule, the electrons being supplied by NADPH. The methyl group at the 14α position of the bound substrate is first oxidized to acetaldehyde moiety and subsequently cleaved as formate.

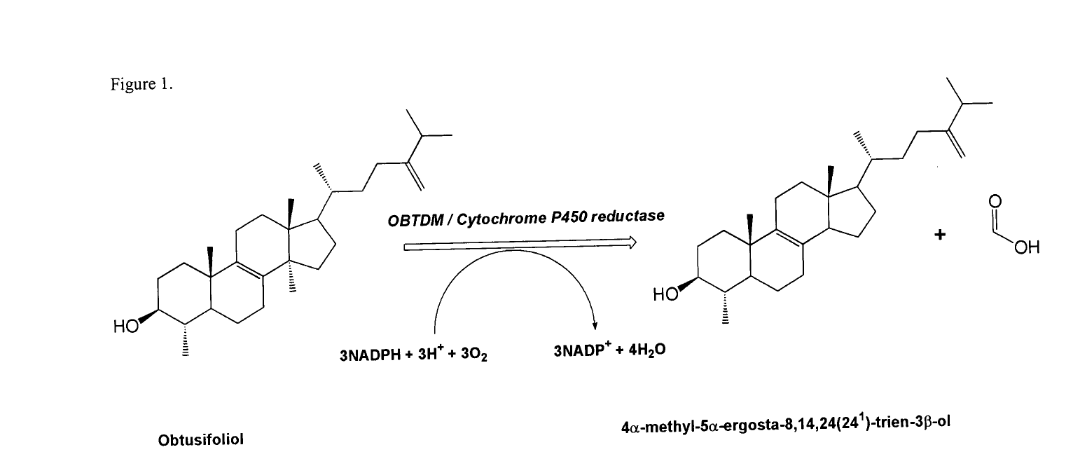

FIGS. 2A-2C. FIG. 2A depicts binding of the nitrogen group of the imidazole or triazole moiety of a type II inhibitor to the central heme iron of a OBT-DM protein, causing a red shift in the Soret band (spectrum shown in FIG. 2B) the magnitude of which is proportional to the strength of the interaction. FIG. 2B depicts the absorbance spectrum (the y axis is absorbance and the x axis are wavelengths ranging from 350-500 nm) of an OBT-DM protein in the presence (solid line) and absence (dashed line) of a bound type II inhibitor. FIG. 2C shows the difference spectrum for FIG. 2B, exhibiting a trough at 413 nm and a peak at 432 nm.

FIGS. 3A-3J. FIG. 3A-J is a depiction of the structures of various type II inhibitors of OBT-DM. All of the compounds contain a triazole or imidazole moiety. The compound Myclobutanil (FIG. 3A) is sold under the trade names Systhane, Syseant, Nova, and Rally and used in agriculture to protect crops against systemic fungal infection. The compound 3-aminotriazole (FIG. 3H) is used as a non-specific herbicide and sold under the trade names Weedazole, Cytrol, Amitrol, Vorox, and Domatol.

FIG. 4. FIG. 4 is a scatter plot of the difference in absorbance (A413-A432) versus column well number (384-well plate format) for the results of an assay for the identification of OBT-DM inhibitors. The assay was performed using 0.4 mg/ml OBTDM in 50 mM HEPES, pH 7.2, and 0.01% Tween 20 in all wells plus 10 μM Ketoconazole (positive control) in 50 mM HEPES, pH 7.2, and 0.01% Tween 20 in plate rows A-H; 10 μM compounds numbered 1-24 in 50 mM HEPES, pH 7.2, and 0.01% Tween 20 in row I; and 50 mM HEPES, pH 7.2, and 0.01% Tween 20 in rows K-P (negative control). The plate was incubated at room temperature for 20 minutes, after which it was read at 413 nm using 432 nm as the reference wavelength on a Tecan Safire spectrophotometric reader. The scatter plot displays the different type II inhibitor test compounds numbered as 1-11 and non-inhibitor compounds numbered as 12-24. The identity of the test compounds is as follows: 4-triazolephenol (1), Fluconazole (2), 4-phenylimidazole (3), Clotrimazole (4), Fluquinconazole (5), 4-imidazolephenol (6), Myclobutanil (7), Prochloraz (8), Econazole (9), 3-amino-1,2,4-triazole (10), itraconazole (11), 4-hydroxyphenoxyacetic acid (12), 3-bromo-4-fluorocinnamic acid (12), 4-dimethylaminobenzaldehyde (14), 2-nitrophenol (15), tricarballylic acid (16), napthyleneacetic acid (17), benzoic acid (18), 3-iodopropionic acid (19), caffeic acid (20), 5-hydroxyindoleacetic acid (21), 5-bromoethylamine (22), phenylmethylsulfonyl fluoride (23) and trans-DL-1,2-cyclopentanedicarboxylic acid (24).

### DETAILED DESCRIPTION OF THE INVENTION

Definitions

A term not otherwise defined is intended to have its ordinary meaning.

The term “binding” refers to a noncovalent interaction that holds two molecules together. For example, two such molecules could be an enzyme and an inhibitor of that enzyme. Noncovalent interactions include hydrogen bonding, ionic interactions among charged groups, van der Waals interactions and hydrophobic interactions among nonpolar groups. One or more of these interactions mediates the binding of two molecules to each other.

As used herein, the term “cDNA” means complementary deoxyribonucleic acid.

As used herein, the term “GUS” means β-glucouronidase.

The term “herbicide”, as used herein, refers to a compound useful for killing or suppressing the growth of at least one plant, plant cell, plant tissue or seed.

The term “inhibitor,” as used herein, refers to a chemical substance that eliminates or substantially reduces OBT-DM enzymatic activity, wherein “substantially” means a reduction at least as great as the standard deviation for a measurement, preferably a reduction by 50%, more preferably a reduction of at least one magnitude, i.e. to 10%.

As used herein, the term “LB” means Luria-Bertani media.

As used herein, the term “Ni-NTA” refers to nickel sepharose.

As used herein, the term “OBT-DM” refers to a polypeptide that catalyzes the 14α-demethylation of sterol substrates that have a 14α-methyl group and a Δ⁸⁽⁹⁾-bond to produce the corresponding sterol Δ^{8(9),14(15)}-dienes. The OBT-DM polypeptides of the invention include, but are not limited to, Arabidopsis thalianana OBT-DM (SEQ ID NO:1), Oryza sativan OBT-DM (SEQ ID NO:2), Nicotiniana tabacum OBT-DM (SEQ ID NO:3), Triticum aestivum OBT-DM (SEQ ID NO:4), Sorghum bicolor OBT-DM (SEQ ID NO:5), Saccharomyces cerevisiae OBT-DM (SEQ ID NO:6), Candida glabratan OBT-DM (SEQ ID NO:7), Uncinula necator OBT-DM (SEQ ID NO:8), Cunninghamella elegans OBT-DM (SEQ ID NO:9), Mycobacterium tuberculosis OBT-DM (SEQ ID NO:10), and Homo sapiens OBT-DM (SEQ ID NO:11).

As used herein, the term “PCR” means polymerase chain reaction.

“Plant” refers to whole plants, plant organs and tissues (e.g., stems, roots, ovules, stamens, leaves, embryos, meristematic regions, callus tissue, gametophytes, sporophytes, pollen, microspores and the like) seeds, plant cells and the progeny thereof.

By “plant OBT-DM” is meant an OBT-DM polypeptide that is naturally occurring in at least one plant species. The OBT-DM is from any plant, including monocots, dicots, C3 plants, C4 plants and/or plants that are classified as neither C3 nor C4 plants.

By “polypeptide” is meant a chain of at least four amino acids joined by peptide bonds. The chain is linear, branched, circular or combinations thereof. The polypeptides may contain amino acid analogs and other modifications, including, but not limited to glycosylated or phosphorylated residues.

As used herein, the term “SDS-PAGE” means sodium dodecyl sulfate-polyacrylimide gel electrophoresis.

The term “specific binding” refers to an interaction between OBT-DM and a molecule or compound, wherein the interaction is dependent upon the primary amino acid sequence or the conformation of OBT-DM. Compounds that do not specifically bind to an OBT-DM polypeptide do not affect the absorbance at 413 nm or 432 nm of the OBT-DM polypeptide.

The present invention provides methods for identifying compounds that inhibit OBT-DM protein activity. OBT-DM enzyme activity comprises the 14α-demethylation of sterol substrates that have a 14α-methyl group and a Δ⁸⁽⁹⁾-bond to produce the corresponding sterol Δ^{8(9),14(5)}-dienes. Identification of compounds that inhibit OBT-DM enzyme activity in the methods of the invention involves measurement of the amplitude of the difference in absorbance of the OBT-DM protein at 413 nm and at 432 nm in the presence and the absence of compounds. Compounds identified by the methods of the invention as being inhibitors of OBT-DM protein activity are useful as antibiotics and herbicides, especially as herbicides.

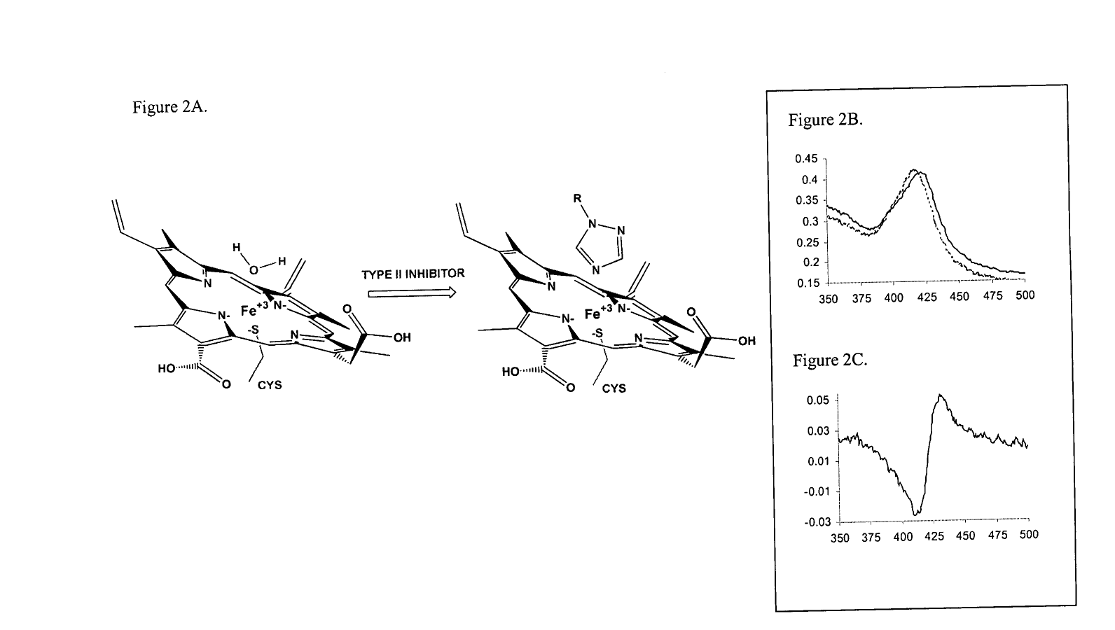

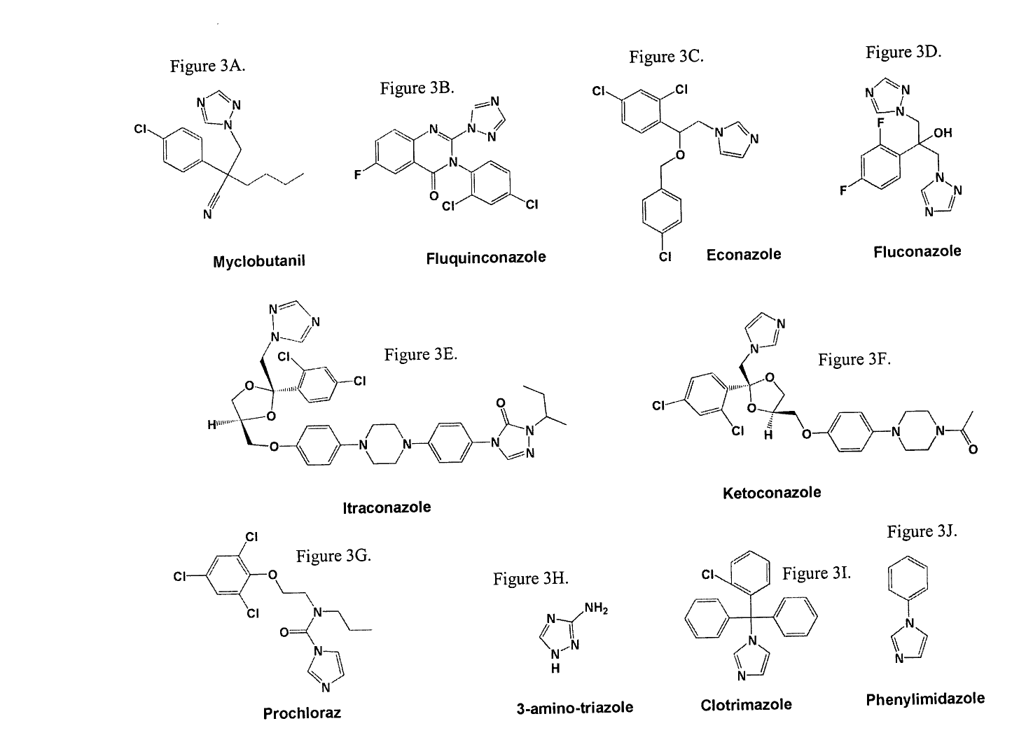

Commercial CYP51 or OBT-DM inhibitors are based on a triazole or imidazole moiety (FIGS. 3A-3J). The nitrogen of these five-member heterocyclics coordinate with heme iron as a sixth ligand causing the latter to shift from high to low spin state, which is reflected by an accompanying red shift in the Soret band (FIGS. 2A-2C). The degree of the shift is a measure of the strength of interaction between the inhibitor compound and the OBT-DM protein. The difference spectrum (spectrum before the addition of the inhibitor subtracted from the one taken after the addition) shows a trough at 413 nm and a peak at 432 nm and is referred to as a “type II spectrum” (Lamb et al., supra) to distinguish it from the spectrum obtained as a result of binding of the substrate to the enzyme or “type I spectrum” (Bak et al., supra).

The change in absorbance of an OBT-DM protein upon binding of a type II inhibitor is used to advantage in the methods of the present invention for a screening assay to identify inhibitory compounds. Rather than measuring the absorbance of an OBT-DM/inhibitor complex over a spectrum of wavelengths as described previously (e.g. the spectrum in FIG. 2B ranges from 350-500 nm), the present invention discloses methods for identifying type II inhibitors of OBT-DM by monitoring the absorbance only at 413 nm and 432 nm. Thus, the methods of the present invention enable the concurrent testing of multiple compounds using a high throughput format such as with 96- or 384-well plates.

In one embodiment, the invention provides a method for identifying an inhibitor of an OBT-DM enzyme, comprising: incubating an OBT-DM polypeptide in the presence and absence of a test compound under conditions suitable for OBT-DM activity; and measuring the amplitude of the difference between the absorbance at 432 nm and the absorbance at 413 nm in the presence and the absence of the test compound, wherein an increase in the amplitude in the presence of the test compound indicates that the compound is an OBT-DM inhibitor. In the methods of the invention, incubating the OBT-DM polypeptide in the absence of a test compound includes incubation of the OBT-DM polypeptide with one or more compounds that are known to not specifically bind to the OBT-DM polypeptide. Such compounds that are known not to specifically bind to the OBT-DM polypeptide are herein not considered “test compounds” and do not affect the absorbance at 413 nm or 432 nm of the OBT-DM polypeptide.

The methods of the invention include methods for identifying inhibitors of an OBT-DM enzyme using either individual test compounds or mixtures of test compounds. Thus, another embodiment of the invention provides a method for identifying an inhibitor of an OBT-DM enzyme, comprising: incubating an OBT-DM polypeptide in the presence of at least one test compound under conditions suitable for OBT-DM activity; incubating the OBT-DM polypeptide under the same conditions in the absence of a compound that specifically binds to the OBT-DM; and measuring the amplitude of the difference between the absorbance at 432 nm and the absorbance at 413 nm for both incubations, wherein an increase in the amplitude in the presence of the test compound(s) indicates that at least one of the test compounds is an OBT-DM inhibitor.

The methods of the invention are amenable to high throughput formats. Thus, another embodiment of the invention is a method for the concurrent testing of a plurality of compounds for an ability to inhibit OBT-DM enzyme activity, comprising: incubating a plurality of test compounds in a multi-well format, individually or in mixtures, with an OBT-DM polypeptide under conditions suitable for OBT-DM activity; incubating in at least one of the wells a negative control comprising the OBT-DM polypeptide under conditions suitable for OBT-DM activity with either no test compound or one or more compounds known not to bind specifically to the OBT-DM; measuring for each of the wells the amplitude of the difference between the absorbance at 432 nm and the absorbance at 413 nm; and comparing the amplitude of the difference in absorbance between the wells comprising the test compound(s) and the negative control(s), wherein an increase in the amplitude in the wells comprising the test compound(s), relative to the well comprising the negative control(s), indicates that at least one of the test compounds comprised within is an OBT-DM inhibitor.

In another embodiment of the invention, a method is provided for the concurrent testing of a plurality of compounds for an ability to inhibit OBT-DM enzyme activity, comprising: incubating a plurality of test compounds in a multi-well format, individually or in mixtures, with an OBT-DM polypeptide under conditions suitable for the OBT-DM activity, wherein at least one of the wells is a negative control comprising either no test compound or one or more compounds known not to bind specifically to the OBT-DM; measuring with a spectrophotometer the absorbance at 413 nm for each of the wells, the absorbance at 413 nm being measured using 432 nm as a reference wavelength on the spectrophotometer; and comparing the absorbance at 413 nm between the wells comprising the test compound(s) and the negative control(s), wherein a decrease in the absorbance at 413 nm in the wells comprising the test compound(s), relative to the negative control(s), indicates that at least one of the test compounds comprised within is an OBT-DM inhibitor. In this particular embodiment of the invention, the absorbance at 432 nm is automatically subtracted from the absorbance at 413 nm by the spectrophotometer so that the absorbance reading for each of the wells of the plate reflects the amplitude of the difference in absorbance at the respective wavelengths.

In one embodiment of the invention, the OBT-DM has the amino acid sequence of a naturally occurring OBT-DM found in a plant, animal or microorganism. In another embodiment of the invention, the OBT-DM has an amino acid sequence derived from a naturally occurring sequence. In another embodiment, the OBT-DM is a plant OBT-DM. In another embodiment, the plant OBT-DM is a from a dicot plant. In another embodiment, the plant OBT-DM is a from a monocot plant. In another embodiment, the OBT-DM is an Arabidopsis OBT-DM which includes, but is not limited to, Arabidopsis arenosa, Arabidopsis bursifolia, Arabidopsis cebennensis, Arabidopsis croatica, Arabidopsis griffithiana, Arabidopsis halleri, Arabidopsis himalaica, Arabidopsis korshinskyi, Arabidopsis lyrata, Arabidopsis neglecta, Arabidopsis pumila, Arabidopsis suecica, Arabidopsis thaliana and Arabidopsis wallichii. In another embodiment, the OBT-DM is a fungal OBT-DM. In another embodiment, the OBT-DM is a human OBT-DM.

Examples of particular OBT-DM polypeptides of the invention include, but are not limited to Arabidopsis thalianana OBT-DM (SEQ ID NO:1; Accession No. NP_{—}172633), Oryza sativa OBT-DM (SEQ ID NO:2; Accession No. NP_{—}912108), Nicotiniana tabacum OBT-DM (SEQ ID NO:3; Accession No. AAL40888), Triticum aestivum OBT-DM (SEQ ID NO:4; Accession No. P93596), Sorghum bicolor OBT-DM (SEQ ID NO:5; Accession No. P93846), Saccharomyces cerevisiae OBT-DM (SEQ ID NO:6; Accession No. P10614), Candida glabrata OBT-DM (SEQ ID NO:7; Accession No. P50859), Uncinula necator OBT-DM (SEQ ID NO:8; Accession No. O14442), Cunninghamella elegans OBT-DM (SEQ ID NO:9; Accession No. Q9UVC3), Mycobacterium tuberculosis OBT-DM (SEQ ID NO:10; Accession No. P77901), and Homo sapiens OBT-DM (SEQ ID NO:11; Accession No. Q16850).

In various embodiments, the OBT-DM can be from barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonum lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium, and the like.

Polypeptides consisting essentially of SEQ ID NO:1-11 are also useful in the methods of the invention. For the purposes of the present invention, a polypeptide consisting essentially of any one of SEQ ID NOS:1-11 has at least 90% sequence identity with the particular SEQ ID NO:1-11 and at least 10% of the activity of the SEQ ID NO:1-11. For example, a polypeptide consisting essentially of SEQ ID NO:1 has at least 90% sequence identity with Arabidopsis OBT-DM (SEQ ID NO:1) and at least 10% of the activity of SEQ ID NO:1. A polypeptide consisting essentially of SEQ ID NO:1 has at least 90%, 91%, 92%, 93%, 94%, 95%, 96%, 97%, 98%, or 99% sequence identity with SEQ ID NO:1 and at least 25%, 50%, 75%, or 90% of the activity of Arabidopsis OBT-DM (SEQ ID NO:1). Examples of polypeptides consisting essentially of any one of SEQ ID NOS:1-11 include, but are not limited to, polypeptides having the amino acid sequence of any one of SEQ ID NOS:1-11 with the exception that one or more of the amino acids of any particular SEQ ID NO are substituted with structurally similar amino acids providing a “conservative amino acid substitution.” Conservative amino acid substitutions are well known to those of skill in the art. Particular examples of polypeptides consisting essentially of SEQ ID NO:1 include polypeptides having 1, 2, or 3 conservative amino acid substitutions relative to SEQ ID NO:1.

Other examples of polypeptides consisting essentially of any one of SEQ ID NOS:1-11 include polypeptides having the sequence of any one of SEQ ID NOS:1-11, but with truncations at either or both the 3′ and the 5′ end of the particular SEQ ID NO. For example, polypeptides consisting essentially of SEQ ID NO:1 include polypeptides having 1, 2, or 3 amino acids residues removed from either or both 3′ and 5′ ends relative to SEQ ID NO:1. In addition, OBT-DM polypeptides consisting essentially of SEQ ID NO:1 can be fusion proteins, such as SEQ ID NO:12, in which an OBT-DM polypeptide is fused with another polypeptide or amino acid sequence to aid in secretion and/or purification, as is known to those of skill in the art. SEQ ID NO:12 is an amino-terminal OBT-DM fusion polypeptide (6-His tag, thrombin cleavage site, S-tag, and enterokinase fused to Arabidopsis thaliana OBT-DM, in that order, where the first 34 amino acids of the Arabidopsis thaliana OBT-DM protein have been deleted).

Test compounds that are identified by the methods of the present invention to be inhibitors of OBT-DM activity are further tested as herbicides by direct application to a plant or plant cell, or expression therein, and monitoring the plant or plant cell for changes or decreases in growth, development, viability or alterations in gene expression. A decrease in growth occurs where the herbicide candidate causes at least a 10% decrease in the growth of the plant or plant cells, as compared to the growth of the plants or plant cells in the absence of the herbicide candidate. A decrease in viability occurs where at least 20% of the plants cells, or portions of the plant contacted with the herbicide candidate, are nonviable. Preferably, the growth or viability will be decreased by at least 40%. More preferably, the growth or viability will be decreased by at least 50%, 75%, or at least 90% or more. Methods for measuring plant growth and cell viability are known to those skilled in the art. It is possible that a test compound may have herbicidal activity only for certain plants or certain plant species.

For use in the screening assays of the invention, OBT-DM protein and derivatives thereof may be isolated from a plant or may be recombinantly produced in and isolated from a plant, bacteria or eukaryotic cell culture. Preferably OBT-DM proteins are produced using a baculovirus, E. coli or yeast expression system. Methods for generating isolated OBT-DM polypeptide are found, for example, in Bak et al., supra and herein at Examples 1 and 2. Other methods for the purification of OBT-DM proteins and polypeptides are known to those skilled in the art.

Chemicals, compounds, or compositions identified by the above methods as modulators of OBT-DM activity are useful for controlling plant growth. For example, compounds that inhibit plant growth are applied to a plant to prevent plant growth. Thus, the invention provides a method for inhibiting plant growth, comprising contacting a plant with a compound identified by the methods of the invention as having herbicidal activity.

Test compounds identified by the methods of the invention as herbicide candidates are useful for controlling the growth of undesired plants, including monocots, dicots, C3 plants, C4 plants, and plants that are neither C3 nor C4 plants. Examples of undesired plants include, but are not limited, to barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonum lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium, and the like.

### EXPERIMENTAL

### EXAMPLE 1

### Cloning of a cDNA Encoding OBT-DM Protein

Total RNA was collected from 14-day-old Arabidopsis thaliana seedlings using published protocols, and reagents (Trizol) from Life Technologies, Inc. (Rockville, Md.). One μl of 10 μM custom oligo, TTA AGA AAG CTG GCG CCT CTT (SEQ ID NO:13), was incubated with 1 μg of total RNA in a reverse transcriptase polymerase chain reaction (RT-PCR) (Invitrogen Corp., Carlsbad, Calif.) according to the manufacturer's recommendations. The OBT-DM cDNA, containing a 102 nucleotide N-terminal signal peptide deletion, was then selectively amplified by PCR with the primer pair CCG GGA TCC AAG AAG AAG CGT CTT CCT CCT (SEQ ID NO:14) and CCG CTC GAG TTA AGA AAG CTG GCG CCT CTT (SEQ ID NO:15). The resulting PCR product, and plasmid pET30a(+) (Novagen, Madison, Wis.) were digested with restriction endonucleases BamHI and XhoI as directed by the manufacturer (New England Biolabs, Beverly, Mass.). Ligation of the two linear DNAs into the resulting recombinant clone pET30a-CYP51 was accomplished by following instructions included with T4 DNA ligase (New England Biolabs, Beverly, Mass.). The integrity of the above clone was verified by DNA sequence analysis to confirm the sequence set forth in SEQ ID NO:12.

### EXAMPLE 2

### Expression and Isolation of Recombinant OBT-DM Protein

The recombinant plasmid pET30a-tCYP-N polyhis was used to transforme E. coli Rosetta DE3 pLysS (Novagen Cat. No. 70956-4) competent cells following manufacturer's instructions. Transformed E. coli was grown overnight at 37° C. in 1 liter of LB medium containing 50 μg/ml kanamycine and 34 μg/ml chloramphenicol. The overnight culture was used to inoculate 36 liters of LB medium containing the two antibiotics in the fermenter. Cells were grown at 37° C. at 80% oxygen saturation to an optical density of 0.8 at which point the medium was made 1 mM in IPTG and 0.5 mM in δ-aminolevulinic acid (to induce heme production) (Jackson et al., 277 J. Biol. Chem. 46959-65 (2002)). The fermenter was allowed to cool to ambient temperature and induction of the recombinant protein was allowed to take place for 15 hours. Cells were harvested by micro-filtration in conjunction with centrifugation. The resulting pellet was resuspended in 500 ml BugBuster (Novagen Cat. No. 70584) containing 600 μl Benzonase (Novagen Cat. No. 70746) and 6 tablets of an EDTA free complete protease inhibitor (Roche Cat. No. 1873580). Cell lysis was allowed to take place at room temperature for 30 minutes. Cellular debris was removed by centrifugation at 20,000×g for 30 minutes. The supernatant was made 5 mM in imidazole and passed through 40 ml of a Ni⁺²-NTA matrix (Qiagen Cat. No. 1018142). The column was washed with five column volumes of 20 mM imidazole in 50 mM Tris, pH 8, containing 300 mM NaCl, and then with 50 mM imidazole in 50 mM Tris, pH 8, containing 300 mM NaCl. The OBT-DM protein was eluted with 500 mM imidazole in 50 mM Tris, pH 8, containing 300mM NaCl. The eluted protein was buffer exchanged into 50 mM HEPES, pH 7.2, by gel filtration on Sephadex G25. The collected supernate contained soluble OBT-DM fusion protein (SEQ ID NO:12), as determined by Coomossie and western blot analysis.

### EXAMPLE 3

### Assay for the Identification of Inhibitors of OBT-DM Enzyme Activity

The optimum wavelength useful for identifying inhibitors of OBT-DM activity was determined as follows. An OBT-DM solution (0.25 mg/ml) in 50 mM HEPES, pH 7.2, was placed in a micro-quartz cuvette with 1 cm path length. The solution was used to blank a Hewlett Packard 8453 spectrometer. Then 1 μL of a 1 mM solution of type II inhibitor in 50% (v/v) DMSO ethanol was added and the spectrum was acquired between the 300 and 500 nm region. All of the inhibitors studied by this method (i.e. Ketoconazole, Fluconazole, Clotrimazole, phenylimidazole) resulted in minimum and maximum absorbance at 413 nm and 432 nm, respectively. This result also translated to a Tecan Safire spectrophotometric reader, and therefore, these wavelengths were chosen as a measure of type II difference spectrum. A maximum magnitude of the signal was observed with Ketoconazole, hence this inhibitor was chosen for use as a positive control in assay development.

An assay for the identification of inhibitors of OBT-DM activity was performed as follows. The assay was set up in a 384-well plate format and initiated by the addition of 40 μl of 20 μM Ketoconazole (ICN Biomedicals, Inc., Irvine, Calif.) in 50 mM HEPES, pH 7.2, 0.01% Tween 20, to rows A-H to a final concentration of 10 μM (positive control) and 40 μl of 50 mM HEPES, pH 7.2, 0.01% Tween 20, to rows K-P (negative control). Test compounds were added to a concentration of 10 μM in row I by manual addition of 40 μl of a 20 μM solution (compounds assigned numbers from 1-24). Then 40 μl of an 0.8 mg/ml solution of OBT-DM protein in 50 mM HEPES, pH 7.2, 0.01% Tween 20 (from Example 2) was added to all wells to a final concentration of 0.4 mg/ml via multidrop. The plate was incubated at room temperature for 20 minutes, after which it was read at 413 nm using 432 nm as the reference wavelength on a Tecan Safire spectrophotometric reader.

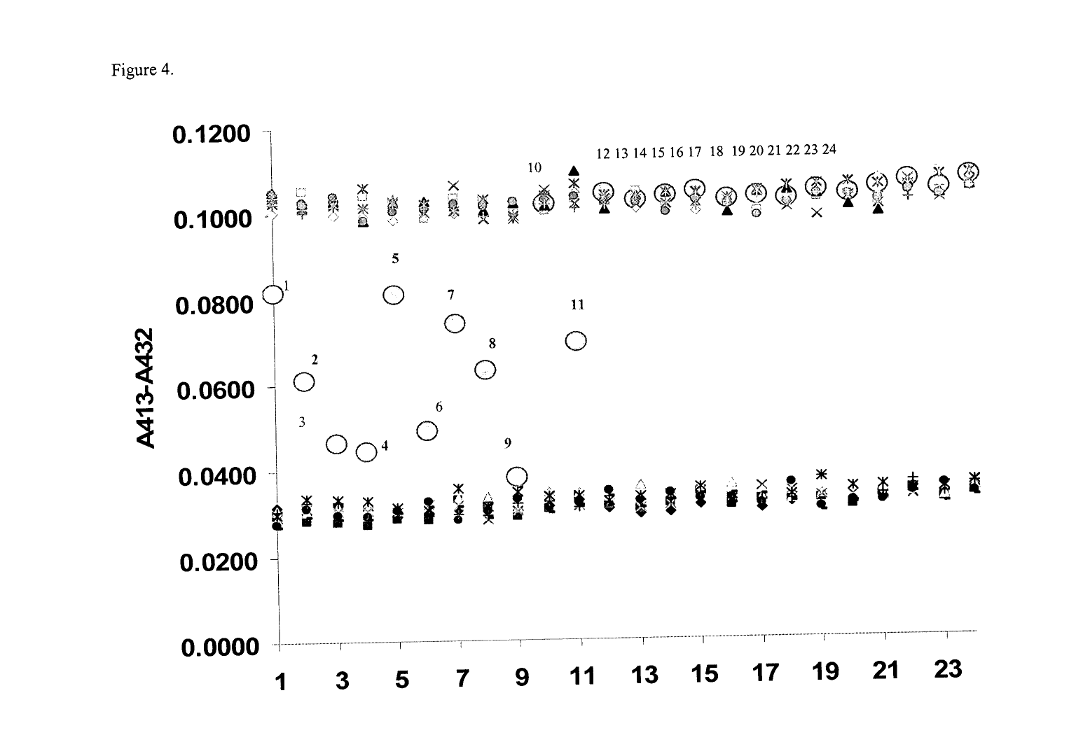

The results of the assay are displayed in FIG. 4. The identity of the type II inhibitor compounds (1-11) and non-inhibitor compounds (12-24) used in the assay are as follows: 4-triazolephenol (1), Fluconazole (2), 4-phenylimidazole (3), Clotrimazole (4), Fluquinconazole (5), 4-imidazolephenol (6), Myclobutanil (7), Prochloraz (8), Econazole (9), 3-amino-1,2,4-triazole (10), itraconazole (11), 4-hydroxyphenoxyacetic acid (12), 3-bromo-4-fluorocinnamic acid (12), 4-dimethylaminobenzaldehyde (14), 2-nitrophenol (15), tricarballylic acid (16), napthyleneacetic acid (17), benzoic acid (18), 3-iodopropionic acid (19), caffeic acid (20), 5-hydroxyindoleacetic acid (21), 5-bromoethylamine (22), phenylmethylsulfonyl fluoride (23) and trans-DL-1,2-cyclopentanedicarboxylic acid (24). The wells containing 10 μM Ketoconazole as a positive control have absorbance readings that fall between 0.02 and 0.04, and the negative control wells have absorbance readings between 0.09 and 0.12. In contrast to the non-inhibitory compounds (12-24), each of the type II inhibitor compounds (1-11), with the exception of number 10, have absorbance readings below that of the negative control wells (compound number 10 has an equilibrium inhibitory constant that is greater than 10 μM which accounts for the negative result). The results indicate the ability of the assay to enable one to distinguish between inhibitory and non-inhibitory OBT-DM compounds.

Although the invention has been described with respect to a preferred embodiment thereof, it is also to be understood that it is not to be so limited as changes and modifications can be made therein which are within the full intended scope of this invention as defined by the appended claims.

1. A method for identifying inhibitors of an Obtusifoliol 14α-demethylase (OBT-DM) enzyme, comprising:
a) incubating an OBT-DM polypeptide in the presence and absence of a test compound under conditions suitable for OBT-DM activity; and
b) measuring the amplitude of the difference between the absorbance around 432 nm and the absorbance around 413 nm in the presence and in the absence of the test compound, wherein an increase in the amplitude in the presence of the test compound indicates that the compound is an OBT-DM inhibitor.

2. A method for identifying inhibitors of an OBT-DM enzyme, comprising:
a) incubating an OBT-DM polypeptide in the presence of at least one test compound under conditions suitable for OBT-DM activity;
b) incubating the OBT-DM polypeptide under the same conditions as part (a) either in the absence of the test compound(s) or in the presence one or more compounds known not to bind specifically to the OBT-DM; and
c) measuring the amplitude of the difference between the absorbance around 432 nm and the absorbance around 413 nm for the incubations of parts (a) and (b), wherein a relative increase in the amplitude in the presence of the test compound(s), indicates that at least one of the test compounds is an OBT-DM inhibitor.

3. The method of claim 1, wherein the OBT-DM is a plant OBT-DM.

4. The method of claim 2, wherein the plant is a dicot.

5. The method of claim 2, wherein the plant is a monocot.

6. The method of claim 2, wherein the OBT-DM is an Arabidopsis OBT-DM.

7. The method of claim 2, wherein the OBT-DM is SEQ ID NO:12.

8. The method of claim 2, wherein the OBT-DM is an OBT-DM polypeptide consisting essentially of SEQ ID NO:1.

9. The method of claim 1, wherein the OBT-DM is a fungal OBT-DM.

10. The method of claim 1, wherein the OBT-DM is a human OBT-DM.

11. A method for the concurrent testing of a plurality of compounds for the ability to inhibit OBT-DM enzyme activity, comprising:
a) incubating a plurality of test compounds in a multi-well format, individually or in mixtures, with an OBT-DM polypeptide under conditions suitable for the OBT-DM activity, wherein at least one of the wells is a negative control comprising either no test compound or one or more compounds known not to bind specifically to the OBT-DM;
b) measuring for each of the wells, the amplitude of the difference between the absorbance around 432 nm and the absorbance around 413 nm; and
c) comparing the amplitude of the difference in absorbance between the wells comprising the test compound(s) and the negative control(s), wherein an increase in the amplitude for the wells comprising the test compound(s), relative to the wells comprising the negative control(s), indicates that at least one of the test compounds comprised within is an OBT-DM inhibitor.

12. The method of claim 10, wherein the OBT-DM is a plant OBT-DM.

13. The method of claim 11, wherein the plant is a dicot.

14. The method of claim 11, wherein the plant is a monocot.

15. The method of claim 11, wherein the OBT-DM is an Arabidopsis OBT-DM.

16. The method of claim 11, wherein the OBT-DM is SEQ ID NO:12.

17. The method of claim 11, wherein the OBT-DM is an OBT-DM polypeptide consisting essentially of SEQ ID NO:1.

18. The method of claim 10, wherein the OBT-DM is a fungal OBT-DM.

19. The method of claim 10, wherein the OBT-DM is a human OBT-DM.

20. A method for the concurrent testing of a plurality of compounds for the ability to inhibit OBT-DM enzyme activity, comprising:
a) incubating a plurality of test compounds in a multi-well format, individually or in mixtures, with an OBT-DM polypeptide under conditions suitable for the OBT-DM activity, wherein at least one of the wells is a negative control comprising either no test compound or one or more compounds known not to bind specifically to the OBT-DM;
b) measuring with a spectrophotometer the absorbance at 413 nm for each of the wells, the absorbance at 413 nm being measured using 432 nm as a reference wavelength on the spectrophotometer; and
c) comparing the absorbance at 413 nm between the wells comprising the test compound(s) and the negative control(s), wherein a decrease in the absorbance at 413 nm in the wells comprising the test compound(s), relative to the negative control(s), indicates that at least one of the test compounds comprised within is an OBT-DM inhibitor.

21. The method of claim 20, wherein the OBT-DM is a plant OBT-DM.

22. The method of claim 21, wherein the plant is a dicot.

23. The method of claim 21, wherein the plant is a monocot.

24. The method of claim 21, wherein the OBT-DM is an Arabidopsis OBT-DM.

25. The method of claim 21, wherein the OBT-DM is SEQ ID NO:12.

26. The method of claim 21, wherein the OBT-DM is an OBT-DM polypeptide consisting essentially of SEQ ID NO:1.

27. The method of claim 20, wherein the OBT-DM is a fungal OBT-DM.

28. The method of claim 20, wherein the OBT-DM is a human OBT-DM.


SEQUENCE LISTING (15 sequences)

SEQ ID NO: 1 - protein, 488 aa, Arabidopsis thaliana
MELDSENKLLKTGLVIVATLVIAKLIFSFFTSDSKKKRLPPTLKAWPPLVGSLIKFLKGPIIMLREEYPKLGSVFTVNLVHKKITFLIGPEVSAHFFKASESDLSQQEVYQFNVPTFGPGVVFDVDYSVRQEQFRFFTEALRVNKLKGYVDMMVTEAEDYFSKWGESGEVDIKVELERLIILTASRCLLGREVRDQLFDDVSALFHDLDNGMLPISVLFPYLPIPAHRRRDRAREKLSEIFAKIIGSRKRSGKTENDMLQCFIESKYKDGRQTTESEVTGLLIAALFAGQHTSSITSTWTGAYLMRYKEYFSAALDEQKNLIAKHGDKIDHDILSEMDVLYRCIKEALRLHPPLIMLMRASHSDFSVTARDGKTYDIPKGHIVATSPAFANRLPHIFKDPDTYDPERFSPGREEDKAAGAFSYIAFGGGRHGCLGEPFAYLQIKAIWSHLLRNFELELVSPFPEIDWNAMVVGVKGNVMVRYKRRQLS

SEQ ID NO: 2 - protein, 507 aa, Oryza sativa
MDHVTSSTIARGAMSWVAATVALLLTTAVILTALQKRKISSPAAAAPPVVRGAGLVRFARAMARDGPLEAIREQQAKLGSVFTAIAPFGLFKVTFLIGPEVSSHFYLAPESEMGQGSIYRFTVPLFGPEVGYAVDPDTRAEQMRLFWDVLKPRSIEARVGAMAEEVQNYFSRWGEQGTVDLKKELEQVLMLIASRCLLGREVRESMVDEVYELFRDLDNGLHLISTMLPYLPTPAHRRRDRARQRLGEIFTEVIRSRRNSGTADNGDDVLQRLIDGRYKDERDLTDVEVVGLLVALVFAGKHSSSSVSTWTGINLLSHPNHLVAVIAEQDRLMASRARTDDDHDRVNYDTVQEMTTLHRCIKEALRLHPPAVAMFRQARKHFTVQTKEGKEYTIPGGHTVMSTILVNHHMPNVYKDPHVFDPSRFARGRGEDKAAGPFSFLAFGAGRHSCAGESFAYTQIKVIWSHLLRNFELKMVSPFPETSWRMVTPEPKGTVMISYRRRNLTCK

SEQ ID NO: 3 - protein, 487 aa, Nicotiana tabacum
MELGDYKILNVGLLLVVTLVVAKLISALIMPRSKKRLPPVIKSWPILGGLLRFLKGPVVMLREEYPKLGSVFTLNLLNKNITFFIGPEVSAHFFKAPETDLSQQEVYQFNVPTFGPGVVFDVDYTIRQEQFRFFTEALRVNKLKGYVDHMVMEAEEYFSKWGDSGEMDLKYELEHLIILTASRCLLGEEVRNKLFEDVSALFHDLDNGMLPISVIFPYLPIPAHRRRDNARKKLAEIFANIIDSRKRTGKAESDMLQCFIDSKYKDGRATTDSEITGLLIAALFAGQHTSSITSTWTGAYLLCNNKYMSAVVDEQKNLMKKHGNKVDHDILSEMEVLYRCIKEVLRLHPPLIMLLRSSHSDFTVKTREGKEYDIPKGHIVATSPAFANRLPHVYKNPDTYDPDRFTPGRDEDKVAGAFSYISFGGGRHGCLGEPFAYLQIKAIWSHLLRNFEFELISPFPEIDWNAMVVGVKGKVMVKYKRRKLSNE

SEQ ID NO: 4 - protein, 453 aa, Triticum aestivum
RPPPTIPGAPVVGGLLRFLRGPIPLIRAEYARLGPVFTVPILTRRITFLIGPDVSAHFFKSNESDMSQQEVYRFNVPTFGPGVVFDVDYQVRQEQFRFFTEALRANKLRSYVDQMVAEAEEYFSKWGESGTVDLKYELEHLIILTASRCLLGREVREKLFDDVSALFHDLDNGMLPISVIFPYLPIPAHRRRDQARTRLAEIFATIIKSRKASGQSEEDMLQCFIDSKYKNGRQTTESEVTGLLIAALFAGQHTSSITSTWTGAYLLKFQQYFAEAVEEQKEVMKRHGDKIDHDILAEMDVLYRCIKEALRLHPPLIMLLRQSHSDFSVTTREGKEFDIPKGHIVATSPAFANRLPHIFKNPDSYDPDRFAAGREEDKVAGAFSYISFGGGRHGCLGEPFAYLQIKAIWTHLLRNFEFELVSPFPENDWNAMVVGIKGEVMVNYKRRKLIVDN

SEQ ID NO: 5 - protein, 492 aa, Sorghum bicolor
MDLADIPQQQRLMAGLALVVATVIFLKLLLSFRSGGGKKRLPPTIPGAPVVGGLVKFMRGPIPMIREQYAALGSVFTVPIITRRITFLIGPEVSAHFFKGNEAEMSQQEVYRFNVPTFGPGVVFDVDYSVRQEQFRFFTEALRANKLRSYVDQMVAEAEEYFSKWGESGTVDLKYELEHLIILTASRCLLGREVREKLFDDVSALFHDLDNGIQPISVLFPYLPIPAHKRRDKARARLAEIFATIIKSRKASGQSEEDMLQCFIDSKYKNGRPTTEGEVTGLLIAALFAGQHTSSITSTWTGAYMLRFKQYFAEAVEEQKDVMKRHGDKIDHDILAEMDVLYRCIKEALRLHPPLIMLLRQSHSDFTVTTKEGKEYDIPKGHIVATSPSFANRLPHIYKNPDSYDPDRFGPGREEDKAAGAFSYISFGGGRHGCLGEPFAYLQIKAIWTHLLRNFEFELVSPFPENDWNAMVVGIKGEVMVNYKRRKLVVDN

SEQ ID NO: 6 - protein, 530 aa, Saccharomyces cerevisiae
MSATKSIVGEALEYVNIGLSHFLALPLAQRISLIIIIPFIYNIVWQLLYSLRKDRPPLVFYWIPWVGSAVVYGMKPYEFFEECQKKYGDIFSFVLLGRVMTVYLGPKGHEFVFNAKLADVSAEAAYAHLTTPVFGKGVIYDCPNSRLMEQKKFVKGALTKEAFKSYVPLIAEEVYKYFRDSKNFRLNERTTGTIDVMVTQPEMTIFTASRSLLGKEMRAKLDTDFAYLYSDLDKGFTPINFVFPNLPLEHYRKRDHAQKAISGTYMSLIKERRKNNDIQDRDLIDSLMKNSTYKDGVKMTDQEIANLLIGVLMGGQHTSAATSAWILLHLAERPDVQQELYEEQMRVLDGGKKELTYDLLQEMPLLNQTIKETLRMHHPLHSLFRKVMKDMHVPNTSYVIPAGYHVLVSPGYTHLRDEYFPNAHQFNIHRWNKDSASSYSVGEEVDYGFGAISKGVSSPYLPFGGGRHRCIGEHFAYCQLGVLMSIFIRTLKWHYPEGKTVPPPDFTSMVTLPTGPAKIIWEKRNPEQKI

SEQ ID NO: 7 - protein, 533 aa, Candida glabrata
MSTENTSLVVELLEYVKLGLSYFQALPLAQRVSIMVALPFVYTITWQLLYSLRKDRPPLVFYWIPWVGSAIPYGTKPYEFFEDCQKKYGDIFSFMLLGRIMTVYLGPKGHEFIFNAKLADVSAEAAYSHLTTPVFGKGVIYDCPNHRLMEQKKFVKGALTKEAFVRYVPLIAEEIYKYFRNSKNFKINENNSGIVDVMVSQPEMTIFTASRSLLGKEMRDKLDTDFAYLYSDLDKGFTPINFVFPNLPLEHYRKRDHAQQAISGTYMSLIKERREKNDIQNRDLIDELMKNSTYKDGTKMTDQEIANLLIGVLMGGQHTSAATSAWCLLHLAERPDVQEELYQEQMRVLNNDTKELTYDDLQNMPLLNQMIKETLRLHHPLHSLFRKVMRDVAIPNTSYVVPRDYHVLVSPGYTHLQEEFFPKPNEFNIHRWDGDAASSSAAGGDEVDYGFGAISKGVSSPYLPFGGGRHRCIGELFAYCQLGVLMSIFIRTMKWRYPTEGETVPPSDFTSMVTLPTAPAKIYWEKRHPEQKY

SEQ ID NO: 8 - protein, 524 aa, Uncinula necator
MYIADILSDLLTQQTTRYGWIFMVTSIAFSIILLAVGLNVLSQLLFRRPYEPPVVFHWFPIIGSTISYGIDPYKFYFDCRAKYGDIFTFILLGKKVTVYLGLQGNNFILNGKLKDVNAEEIYTNLTTPVFGRDVVYDCPNSKLMEQKKFMKTALTIEAFHSYVTIIQNEVEAYINNCVSFQGESGTVNISKVMAEITIYTASHALQGEEVRENFDSSFAALYHDLDMGFTPINFTFYWAPLPWNRARDHAQRTVARTYMNIIQARREEKRSGENKHDIMWELMRSTYKDGTPVPDREIAHMMIALLMAGQHSSSSTSSWIMLWLAARPDIMEELYEEQLRIFGSEKPFPPLQYEDLSKLQLHQNVLKEVLRLHAPIHSIMRKVKNPMIVPGTKYVIPTSHVLISSPGCTSQDATFFPDPLKWDPHRWDIGSGKVLGNDAVDEKYDYGYGLTSTGASSPYLPFGAGRHRCIGEQFATLQLVTIMATMVRFFRFRNIDGKQGVVKTDYSSLFSMPLAPALIGWEKR

SEQ ID NO: 9 - protein, 512 aa, Cunninghamella elegans
MAIVSQISRFITFTIISMGYSVLAVGVALTIHILSQLIVPKNPNEPPNVFSLIPVLGNAVQFGMNPVAFLQECQKKYGDVFTFTMVGKRVTVCLGADGNQFVFNSKQNLSSAAEAYNHMTKYVFGPDVVYDAPHAVFMEQKKFIKAGLNSDCFRQHVPMIVQETEEFFKKFNKPTGFIEAYETFGSLIIYTASRCLMGKEIRASLDGNVAKLYYDLDQGFKPINFIFPNLPLPSYRRRDVACKKMADLYSSIIQRRKDEKDNNNADLLQALMDATYKDGTHIPDHHIAGMMIAVLFGGQHTSATTSAWTILELANRPDIIKALREEQIEKLGSLKADLTFDNLKDLPLLEAAIRETLRLHPPIFQMMRRVVADKIVYEKNGMEIPKGNFICAAPGVTQVDPTYFNEPTTYNPYRWIEKTDPVHQLEQGDDANIDYGFGAVGISSKSPFLPFGAGRHRCIGEQFGYLQLKTVISTFIRTFDFDLDGKSVPKSDYTSMVVVPEHTAKVRYTWRE

SEQ ID NO: 10 - protein, 451 aa, Mycobacterium tuberculosis
MSAVALPRVSGGHDEHGHLEEFRTDPIGLMQRVRDECGDVGTFQLAGKQVVLLSGSHANEFFFRAGDDDLDQAKAYPFMTPIFGEGVVFDASPERRKEMLHNAALRGEQMKGHAATIEDQVRRMIADWGEAGEIDLLDFFAELTIYTSSACLIGKKFRDQLDGRFAKLYHELERGTDPLAYVDPYLPIESFRRRDEARNGLVALVADIMNGRIANPPTDKSDRDMLDVLIAVKAETGTPRFSADEITGMFISMMFAGHHTSSGTASWTLIELMRHRDAYAAVIDELDELYGDGRSVSFHALRQIPQLENVLKETLRLHPPLIILMRVAKGEFEVQGHRIHEGDLVAASPAISNRIPEDFPDPHDFVPARYEQPRQEDLLNRWTWIPFGAGRHRCVGAAFAIMQIKAIFSVLLREYEFEMAQPPESYRNDHSKMVVQLAQPACVRYRRRTGV

SEQ ID NO: 11 - protein, 503 aa, Homo sapiens [sequence omitted]

SEQ ID NO: 12 - protein, 504 aa, Artificial
  Arabidopsis thaliana fusion protein
MHHHHHHSSGLVPRGSGMKETAAAKFERQHMDSPDLGTDDDDKAMADIGSKKKRLPPTLKAWPPLVGSLIKFLKGPIIMLREEYPKLGSVFTVNLVHKKITFLIGPEVSAHFFKASESDLSQQEVYQFNVPTFGPGVVFDVDYSVRQEQFRFFTEALRVNKLKGYVDMMVTEAEDYFSKWGESGEVDIKVELERLIILTASRCLLGREVRDQLFDDVSALFHDLDNGMLPISVLFPYLPIPAHRRRDRAREKLSEIFAKIIGSRKRSGKTENDMLQCFIESKYKDGRQTTESEVTGLLIAALFAGQHTSSITSTWTGAYLMRYKEYFSAALDEQKNLIAKHGDKIDHDILSEMDVLYRCIKEALRLHPPLIMLMRASHSDFSVTARDGKTYDIPKGHIVATSPAFANRLPHIFKDPDTYDPERFSPGREEDKAAGAFSYIAFGGGRHGCLGEPFAYLQIKAIWSHLLRNFELELVSPFPEIDWNAMVVGVKGNVMVRYKRRQLS

SEQ ID NO: 13 - DNA, 21 bp, artificial
  RT-PCR primer
ttaagaaagctggcgcctctt

SEQ ID NO: 14 - DNA, 30 bp, Artificial
  PCR primer
ccgggatccaagaagaagcgtcttcctcct

SEQ ID NO: 15 - DNA, 30 bp, Artificial
  PCR primer
ccgctcgagttaagaaagctggcgcctctt